(19) United States
(12) Patent Application Publication
(10) Pub. No.: US 2020/0391016 A1
(43) Pub. Date: Dec. 17, 2020

(54) Method For Bypassing Defective Portions Of A Heart

(71) Applicant: NXT Biomedical, Irvine, CA (US)

(72) Inventors: Joseph Passman, Costa Mesa, CA (US); Robert S. Schwartz, Inver Grove Heights, MN (US); Robert C. Taft, Orange, CA (US); Stanton J. Rowe, Newport Coast, CA (US); Alexander Siegel, Aliso Viejo, CA (US); Glen Rabito, Lake Forest, CA (US)

(73) Assignee: NXT Biomedical, Irvine, CA (US)

(21) Appl. No.: 16/904,432

(22) Filed: Jun. 17, 2020

Related U.S. Application Data
(60) Provisional application No. 62/862,550, filed on Jun. 17, 2019.

(51) Int. Cl. A61M 27/00; A61F 2/88; A61F 2/95; A61B 18/14

(52) U.S. Cl. CPC A61M 27/002; A61F 2/88; A61B 2018/00577; A61B 18/1492; A61M 2205/0266; A61F 2/95

(57) ABSTRACT

Methods and techniques are disclosed for performing transcatheter procedures on a patient that result in similar blood flow changes to the surgical Blalock-Taussig procedure, the Glenn procedure, and the Fontan procedure. Since these transcatheter procedures do not necessarily involve open chest surgery, they tend to be less traumatic for the patient, quicker and easier to perform, and may result in better patient outcomes.

### RELATED APPLICATIONS

This application claims priority to claims priority to U.S. Provisional Application Ser. No. 62/862,550 filed Jun. 17, 2019 entitled Congenital Defect Shunt, TriCuspid Atresia, which is hereby incorporated herein by reference in its entirety.

### BACKGROUND OF THE INVENTION

Different congenital birth defects can result in poor oxygenation of the blood in young babies and children. Such conditions include tricuspid atresia, pulmonary atresia, pulmonary stenosis, Tetralogy of Fallot, and hypoplastic left heart syndrome. These defects are typically treated with surgical procedures that create shunts to change the patient's blood flow to allow for better oxygenation.

Tricuspid atresia is a congenital heart defect present at birth in which the tricuspid valve between the right atrium 16 and the right ventricle 24 is underdeveloped or entirely missing. Instead, solid tissue 23 creates a barrier between the right atrium 16 and the right and left ventricle 24, 19, blocking blood flow between the two, as seen in FIG. 1. As a result, the right ventricle 24 is typically much smaller and underdeveloped (hypoplastic) than it would otherwise be in a healthy heart 10.

Instead of the normal blood flow through the heart 10, blood flows from the right atrium 16 to the left atrium 18 through a hole in the atrial septum 17 or similar location of the heart 10. This hole is typically either an atrial septal defect 26, patent foramen ovale, or patent ductus arteriosus. As a result, a baby or child with tricuspid atresia is unable to get enough oxygen through its body, creating shortness of breath and blue-tinged skin.

Pulmonary atresia is a rare birth defect in which the pulmonary valve is missing completely or otherwise blocked. Normally, the pulmonary valve allows blood to flow from the right ventricle through the pulmonary artery to the lungs to pick up oxygen. Without this valve, there is no way for blood to move from the right ventricle to the pulmonary artery. Consequently, the central pulmonary artery or each of the right and left branches may be small and unusually arranged.

Pulmonary valve stenosis is a condition in which a baby is born with a pulmonary valve that is too small, narrow, or stiff. Similar to pulmonary atresia, little blood is allowed to pass into the pulmonary artery, causing the right ventricle to pump harder to send blood out to the lungs. Over time, this can cause the right ventricle to thicken and strain the heart.

Tetralogy of Fallot (TOF) is a combination of four congenital heart defects, ventricular septal defect, pulmonary stenosis, right ventricular hypertrophy, and overriding aorta, that changes the normal blood flow to the lungs and through the heart. The result is that not enough blood passes to the lungs and therefore does not carry enough oxygen to other parts of the body.

Hypoplastic left heart syndrome (HLHS) occurs when a baby is born with an abnormality of the heart or heart valves that results in the left side of the heart being smaller and weaker than it should be. This leaves the left side of the heart unable to sufficiently pump blood through the body.

The above-described conditions are commonly treated with one or more (or even all three) surgical procedures to change the flow of blood through the heart and increase oxygenation efficiency. These procedures are generally known as the Blalock-Taussig procedure, the Glenn procedure, and the Fontan procedure, which are shown in FIGS. 2-4 on a heart with tricuspid atresia. Often, the Glenn procedure and later the Fontan procedure are performed on a patient, but especially in cases of tricuspid atresia, all three procedures are often performed in sequence at different ages of the patient.

The Blalock-Taussig procedure is a surgical procedure typically performed shortly after the birth of a child and consists of an anastomosis or shunt 29 between the right pulmonary artery 30 and the right subclavian artery 28, as seen in FIG. 2. In effect, this Blalock-Taussig shunt 29 mimics the role of the ductus arteriosus by allowing blood to flow from a major artery through a connection to the pulmonary artery. This allows more blood to be oxygenated by the lungs and encourages the pulmonary arteries to grow.

The Glenn procedure is a surgical procedure typically performed on patients 4-12 months old and consists of disconnecting the superior vena cava 21 from the heart 10 and then connecting it to the right pulmonary artery 30, as seen in FIG. 3. This arrangement allows blood from the upper body to pass into the pulmonary artery and then to the lungs, without the need to pass through the heart.

The Fontan procedure is a surgical procedure typically performed on patients 18-36 months old and consists of disconnecting the inferior vena cava 12 from the heart 10 and connecting it to the right pulmonary artery 30 via a conduit or shunt 34. The Fontan procedure is typically performed after the Glenn procedure and therefore the superior vena cava 21 is typically already connected to the right pulmonary artery 30. Optionally, the shunt 34 may also include a passage or fenestration 32 into the right atrium 16 to allow some blood to flow directly back to the heart, acting as a “pop-off” valve as the lungs adjust to the extra blood flow. This passage 32 can be later closed via a cardiac catheterization procedure.

Depending on the severity of the congenital defect, only one of these procedures may be needed. However, often all three procedures must be performed on a patient at varying ages in order to temporarily increase blood oxygenation and keep the patient alive until a heart transplant can occur. This may be particularly true for patients with tricuspid atresia, among other conditions.

These procedures can be especially traumatic due to the dramatic changes in the blood flow within a patient, the trauma induced by several major surgeries, and because surgeries are performed on very young patients (e.g., recently born to 36 months old). Therefore, what is needed are less invasive and traumatic procedures to achieve similar results to the Blalock-Taussig procedure, Glenn procedure, and Fontan procedure.

### SUMMARY OF THE INVENTION

The present invention is generally directed to methods of treating heart and vascular defects via transcatheter procedures. These defects include tricuspid atresia, pulmonary atresia, pulmonary stenosis, Tetralogy of Fallot, and hypoplastic left heart syndrome.

The present invention is generally directed to methods and techniques for performing transcatheter procedures on a patient that result in blood flow changes similar to the surgical Blalock-Taussig procedure, the Glenn procedure, and the Fontan procedure. Since these transcatheter procedures do not necessarily involve open chest surgery, they tend to be less traumatic for the patient, quicker and easier to perform, and may result in better patient outcomes.

In one embodiment, an alternate, transcatheter Blalock-Taussig procedure is disclosed in which a shunt scaffold is placed between the aorta and the pulmonary artery to create a passage or shunt. Similar to the original surgical procedure, blood has a path to recirculate back to the lungs and therefore better oxygenate. However, the shunt is instead placed at a location where the aorta and pulmonary artery overlap each other (e.g., at the pulmonary trunk).

In one embodiment, an alternate, transcatheter Glenn procedure is disclosed in which the prior shunt scaffold is closed if present, a new shunt passage is created with a shunt scaffold between the superior vena cava and the right pulmonary artery, and a one-way valve is placed in the superior vena cava. Similar to the original surgical Glenn procedure, this forces blood from an upper portion of the body back into the right pulmonary artery so that it can bypass the heart and flow directly to the lungs for oxygenation.

In one embodiment, an alternate, transcatheter Fontan procedure is disclosed that consists of deploying an elongated covered shunt or stent between the inferior vena cava and the superior vena cava. The top end of the covered stent is preferably placed over the previously places one-way stent valve, if present, preventing it from closing, but below the stent scaffold into the right pulmonary artery.

In one aspect of the present invention, all three procedures are performed, at different times, on a patient. In another aspect of the present invention, only one or more of the three procedures are performed on a patient.

In one aspect of the present invention, the alternate, transcatheter Blalock-Taussig procedure is performed shortly after the birth of a patient (e.g., within about 1 month of birth), the alternate, transcatheter Glenn procedure is performed on a patient that is about 4-12 months old, and the alternate, transcatheter Fontan procedure is performed on a patient that is about 18-36 months old.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 illustrates a heart with tricuspid atresia.

FIG. 2 illustrates a prior art, surgical Blalock-Taussig procedure.

FIG. 3 illustrates a prior art, surgical Glenn procedure.

FIG. 4 illustrates a prior art, surgical Fontan procedure.

FIG. 5 illustrates a procedure for creating a shunt between an aorta and a pulmonary artery according to the present invention.

FIG. 6 illustrates a procedure for creating a shunt between an aorta and a pulmonary artery according to the present invention.

FIG. 7 illustrates blood flow after the procedure of FIGS. 5 and 6 according to the present invention.

FIG. 8 illustrates placement of a shunt scaffold according to the present invention.

FIG. 9 illustrates placement of a shunt scaffold according to the present invention.

FIG. 10A illustrates a shunt scaffold according to the present invention.

FIG. 10B illustrates a shunt scaffold according to the present invention.

FIG. 11 illustrates a shunt scaffold according to the present invention.

FIG. 12 illustrates a shunt scaffold according to the present invention.

FIG. 13A and FIG. 13B illustrate a valve or flow adjustment mechanism for a shunt scaffold according to the present invention.

FIG. 14A and FIG. 14B illustrate a valve or flow adjustment mechanism for a shunt scaffold according to the present invention.

FIG. 15 illustrates a flow diverter mechanism for a shunt scaffold according to the present invention.

FIG. 16 illustrates a flow diverter mechanism for a shunt scaffold according to the present invention.

FIG. 17 illustrates a method of closing a shunt scaffold between the pulmonary artery and the aorta according to the present invention.

FIG. 18 illustrates a method of closing a shunt scaffold between the pulmonary artery and the aorta according to the present invention.

FIG. 19 illustrates blood flow after the procedure from FIGS. 17 and 18 according to the present invention.

FIG. 20 illustrates a method of creating a shunt between an inferior vena cava and a right pulmonary artery according to the present invention.

FIG. 21 illustrates a method of creating a shunt between an inferior vena cava and a right pulmonary artery according to the present invention.

FIG. 22 illustrates a method of delivering a valve to the inferior vena cava according to the present invention.

FIG. 23 illustrates blood flow after performing the methods of FIGS. 20-22 according to the present invention.

FIG. 24 illustrates a method of closing a shunt scaffold according to the present invention.

FIG. 25 illustrates a method of deploying an elongated shunt between the superior vena cava and inferior vena cava according to the present invention.

FIG. 26 illustrates blood flow after performing the methods of FIGS. 24 and 25 according to the present invention.

FIG. 27 illustrates a method of creating a shunt between a pulmonary artery and an aorta, and between a superior vena cava and a right pulmonary artery, according to the present invention.

FIG. 28 illustrates a shunt scaffolding having an inner passage closed off by a sheet, according to the present invention.

FIG. 29 illustrates a method of creating a shunt between a pulmonary artery and an aorta, and between a superior vena cava and a right pulmonary artery, according to the present invention.

FIG. 30 illustrates a method of closing off a shunt between a pulmonary artery and an aorta, according to the present invention.

FIG. 31 illustrates a shunt scaffold with a landing zone in the superior vena cava for later connecting to an elongated covered stent, according to the present invention.

FIG. 32 illustrates an elongated shunt configured to anchor and connect the superior vena cava and the inferior vena cava, according to the present invention.

FIG. 33 illustrates a method of accessing and creating a shunt between an aorta and a pulmonary artery, according to the present invention.

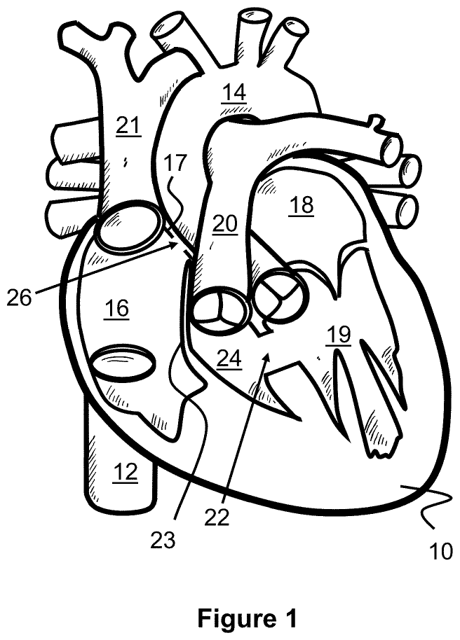

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The present invention is generally directed to methods and techniques for performing transcatheter procedures on a patient that result in similar blood flow changes as with the Blalock-Taussig procedure, the Glenn procedure, and the Fontan procedure. Since these transcatheter procedures do not necessarily involve open chest surgery, they tend to be less traumatic for the patient, quicker and easier to perform, and may result in better patient outcomes.

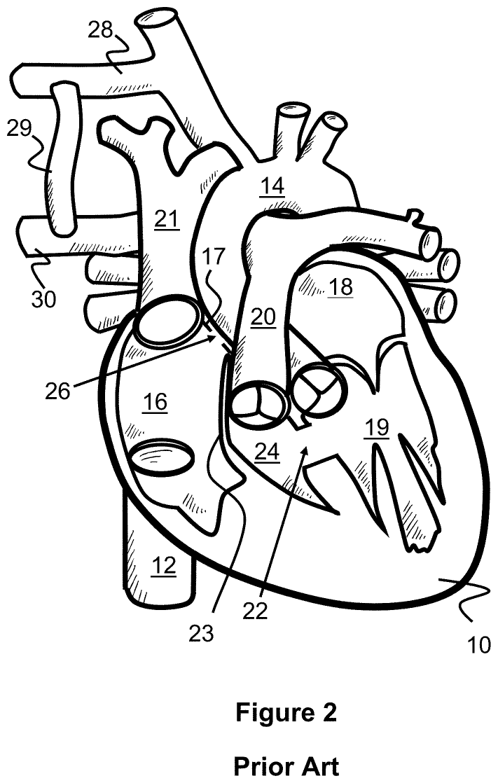

In the original, surgical Blalock-Taussig procedure (FIG. 2) an anastomosis or shunt 29 is surgically connected to the right pulmonary artery 30 and to the right subclavian artery 28. This allows blood from the right subclavian artery 28 to recirculate or pass back to the right pulmonary artery 30 so that it can be further oxygenated by the lungs.

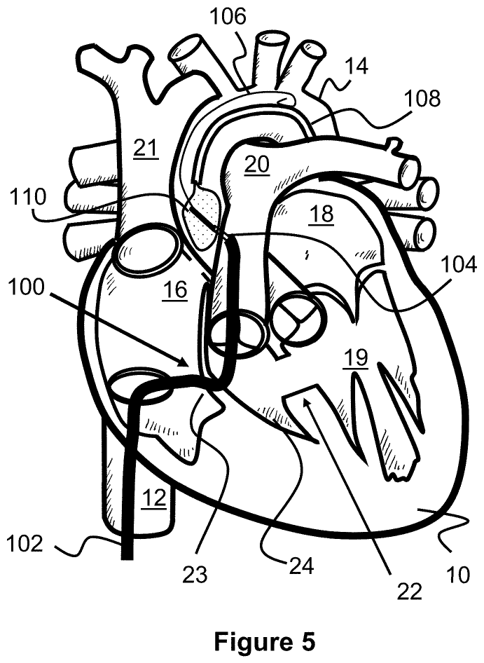

FIGS. 5-7 illustrate an alternate, transcatheter Blalock-Taussig procedure in which a shunt scaffold 130 is placed between the aorta 20 and the pulmonary artery 14 to create a passage or shunt. Similar to the surgical procedure, blood has a path to recirculate and therefore better oxygenate. However, the shunt 130 is instead placed at a location where the aorta 20 and pulmonary artery 14 overlap each other (e.g., at the pulmonary trunk, or about 1-2 cm above the aortic valve and pulmonary valve).

The first step of the present procedure is directed to puncturing a wall of the aorta 14 and a wall of the pulmonary artery 20. FIG. 5 illustrates one example method for creating this puncture with a tissue piercing guidewire 106 (e.g., an RF guidewire) and a target catheter 108, allowing the delivery catheter 100 to later pass through. The target catheter 108 can be advanced into the aorta 14 so that its distal end is located near the crossing of the aorta 14 and pulmonary artery 20. A snare 110 can be advanced out of the distal end of the target catheter 108 to help prevent a far side of the pulmonary artery 20 from being punctured (i.e., the guidewire 106 from passing entirely through the aorta 14).

In one embodiment, the snare 110 is a wire loop that self-expands from a linear shape to a generally rounded loop or circle shape. A puncture resistant material such as a polymer sheet or a finely braided wire mesh can be spread over the loop or circle shape of the snare 110, thereby shielding the opposite side of the pulmonary artery 20 from being punctured. The snare 110 and/or target catheter 108 may include radiopaque markers to assist in aligning the guidewire 106. Additionally or alternately, both the snare 110 and the guidewire 106 may each have magnets that help align the tip of the guidewire 106 with a location of the snare 110.

Next, the tissue piercing guidewire 106 is advanced through the inferior vena cava 12, into the right atrium 16, through the tricuspid valve wall 23, through the pulmonary valve, and into the pulmonary artery 20. The piercing guidewire 106 can be advanced through a wall of the pulmonary valve 20, through a wall of the aorta 14, and into the aorta 14 to contact to snare 110. The delivery catheter 102 can then be advanced over the guidewire 106 until its distal end extends into the puncture within the walls of the pulmonary artery 20 and aorta 14.

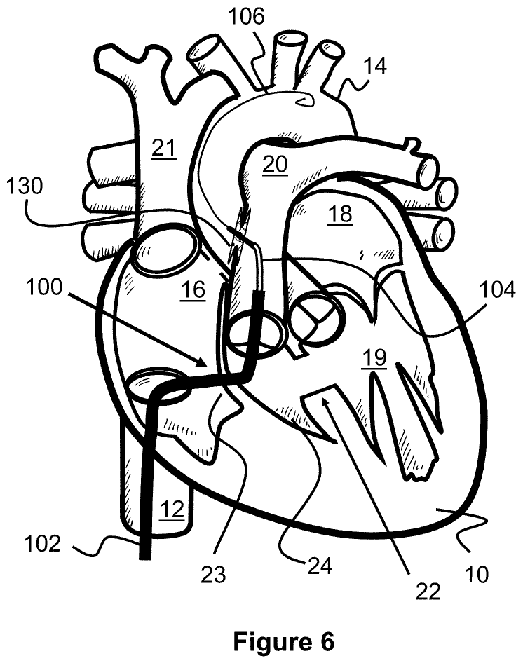

Turning to FIG. 6, a shunt support scaffold 130 can be delivered within and through the walls of the pulmonary artery 20 and the aorta 14. For example, the shunt support scaffold 130 can be radially compressed on an inner catheter portion 104 and maintained in that position by an outer sheath portion 102. When the delivery catheter 100 is positioned at its desired target location within the wall punctures, the outer sheath portion 102 can be proximally withdrawn to allow the shunt support scaffold 130 to radially expand. Portions of the shunt scaffold 130 engage each of the tissue walls and a passage through the scaffold creates a passage between the pulmonary artery 20 and the aorta 14 through which blood can flow.

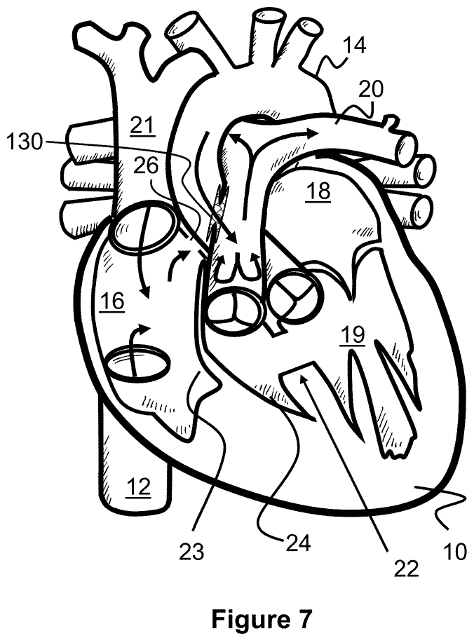

The arrows of FIG. 7 illustrate the subsequent blood flow patterns after the shunt support scaffold 130 has been placed. Some blood from the aorta 14 passes into the pulmonary artery 20 through the shunt support scaffold 130, allowing it to be further oxygenated at the lungs. Blood from the inferior vena cava 12 and superior vena cava 21 continue to pass into the right atrium 16, through the atrial septal defect 26, and into the left atrium 18 and left ventricle 19.

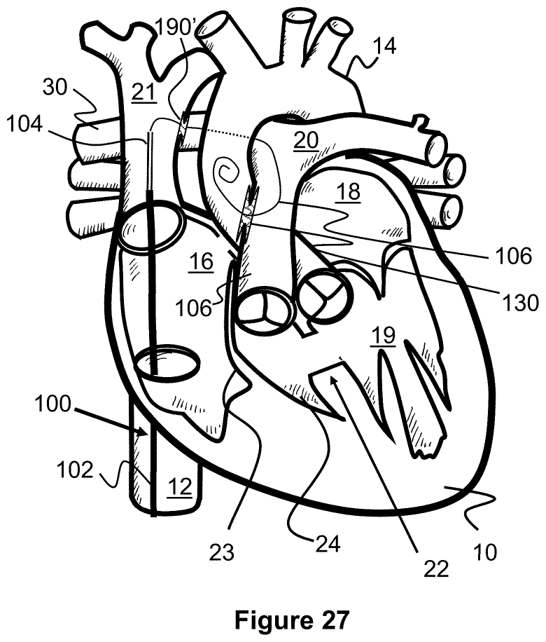

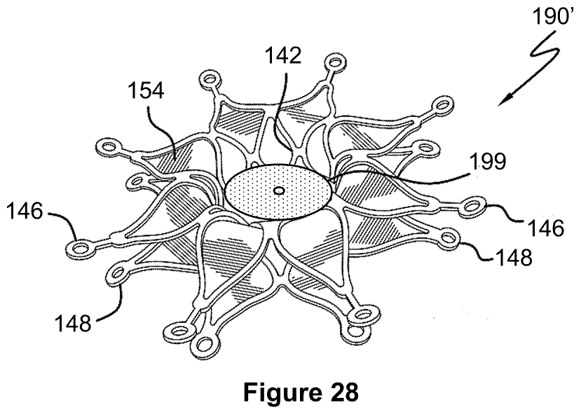

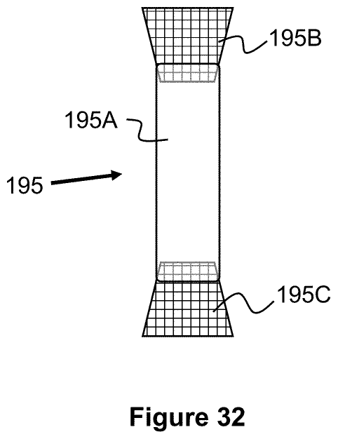

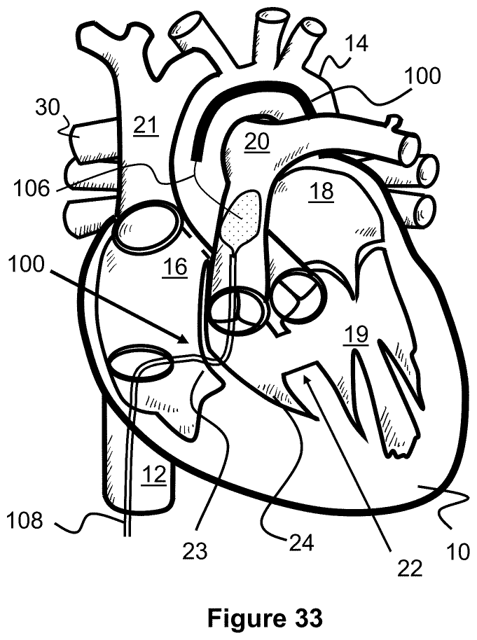

Alternately, the approaches and positions of the delivery catheter 100 and target catheter 108 can be switched, such that the delivery catheter approaches from the aorta 20 and the target catheter approaches from the pulmonary artery 20, as seen in FIG. 32 for example. In such a method, a piercing guidewire 106 can be advanced through the aorta 14, navigating the aortic arch, and then torqued towards the pulmonary artery 20. In some instances, this guidewire path may be somewhat easier to navigate and puncture versus tracking the piercing guidewire 106 into the pulmonary artery 20. Additionally, while the target catheter 108 can be advanced through the tricuspid valve wall 23 and into the pulmonary artery 20, it can alternately be advanced into the superior vena cava 21, into the right pulmonary artery 30, and then down into the trunk of the pulmonary artery 20. This path may also allow for a closed shunt scaffold 190′ to be deployed between the superior vena cava 21 and the right pulmonary artery 20, as discussed later with regard to FIGS. 27 and 28.

In another alternate embodiment, the transcatheter Blalock-Taussig procedure described above may include some surgical procedures that allow the shunt support scaffold 130 to be placed percutaneously. For example, a physician may perform surgical access to the aorta and pulmonary artery, and then from that access, initially puncture the aorta 14 or pulmonary artery 20 with a piercing guidewire 106 and then further pierce through the walls of both vessels at the location of the desired implantation location of the shunt scaffold 130. A delivery catheter 100 can then be advanced over the guidewire 106 and the shunt scaffold 130 can be deployed as described above. It should be noted that this hybrid surgical/percutaneous technique can also be used for any of the other procedures in this specification.

The shunt support scaffold 130 discussed above, or others discussed in this specification, can be self-expanding, expanded via an inflatable balloon on the inner catheter portion 104, or a combination of both. Examples of such devices can be found in U.S. application Ser. No. 16/576,704 filed Sep. 19, 2019 and entitled Method and Technology for Creating Connections and Shunts between Vessels and Chambers of Biological structures, and in U.S. application Ser. No. 16/785,501 filed Feb. 7, 2020 and entitled Rivet Shunt and Method of Deployment, both of which are incorporated by reference.

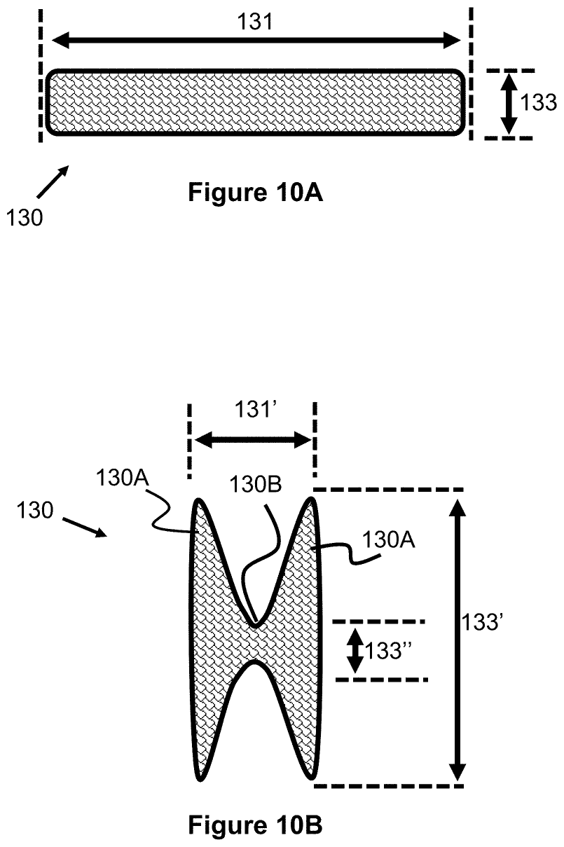

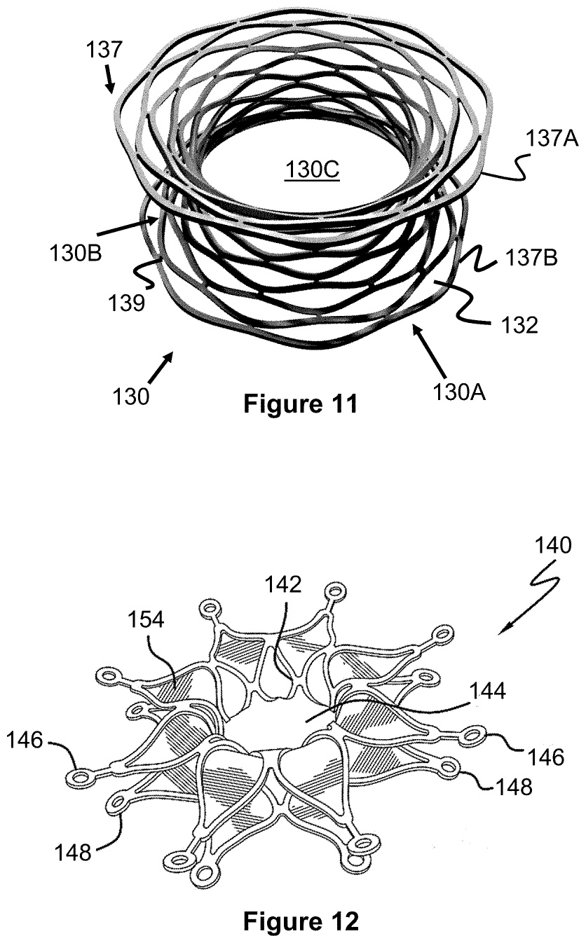

In one embodiment, the scaffold 130 can expand from a relatively uniform cylindrical shape to a shape with a narrowed middle section (e.g., an hourglass shape), as seen in FIGS. 10A, 10B, and 11. In FIG. 10A, the scaffold 130 is shown in a radially compressed configuration having a relatively long length 131 and a relatively small, uniform diameter 133. As the scaffold 130 is deployed and expanded (FIG. 10B), its length substantially decreases to 131′ and its diameter increases. More specifically, end portions 130A increase to a maximum radial diameter of 133′ and then decrease in diameter towards a middle region 130B, which has a diameter of 203″. In one embodiment, the device is composed of a super elastic material such as Nitinol.

In one example, when compressed, the scaffold 130 has a length 131 of about 20 mm and a diameter 133 of about 1.5 mm, and when expanded the shunt 130 has a diameter 133′ of the end portions 130A of about 8 mm and a diameter 133″ of the middle region 130B of about 5 mm.

In another example, when compressed, the scaffold 130 has a length 131 of about 30 mm and a diameter 133 of about 2.2 mm, and when expanded the shunt 130 has a diameter 133′ of the end portion 130A of about 8 mm and a diameter 133″ of the middle region 130B of about 3 mm.

The scaffold 130 includes a plurality of tubular radial bands that are each formed from a plurality of uniform, alternating waves that create the shunt passage 130C. Put another way each radial band 137 comprises a plurality of straight regions 137B joined together to create a pattern of triangular peaks 137A that alternate their longitudinal directions. The peaks 137A of each radial band 137 are aligned with each other and connected via a small, straight portion 139, which effectively creates diamond-shaped cells 132 when radially compressed. As a result of this design, the angle of each peak 137A increases as the scaffold 130 is radially expanded and the radial bands 137 become closer together to each other, which causes longitudinal foreshortening (i.e., a decrease in length of the scaffold 130).

One mechanism for causing the radial flaring of the ends 130A of the scaffold 130 is by creating a pattern of cells 132 in which some cells 132 are longer in their proximal-to-distal length than other cells 132 (i.e., they have longer straight regions 137B). Preferably, cells 132 in the middle of the shunt 130 have the smallest length and each row of cells 132 progressively increase in length the further away from the middle they are. Alternately, larger length cells 132 can be located only near the ends of the shunt 130. Additionally, the radial flaring can be assisted or caused by using an hourglass-shaped balloon 124 (FIGS. 10 and 11) that has larger diameter end portions 124A and 124B relative to the smaller diameter middle portion 124C when inflated.

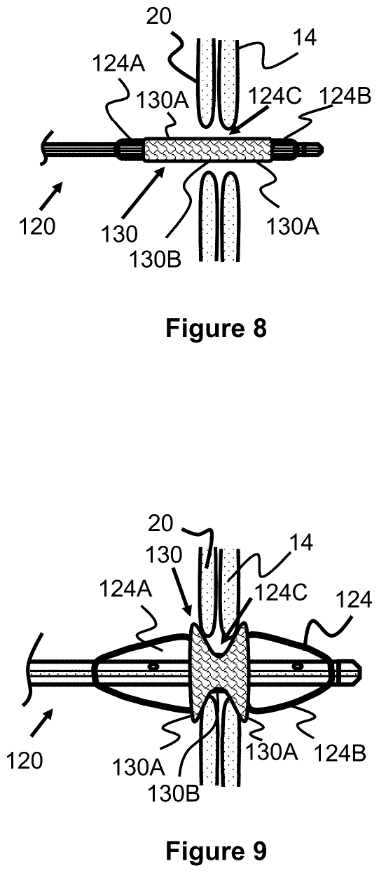

One variation on the delivery technique of the scaffold 130 allows for the passage through the shunt 130C (i.e., the narrowed middle region 130B) to be resized after delivery, if needed. Specifically, the scaffold 130 can be delivered as previously described, but the narrowed middle region 130B is expanded to an initial diameter that is smaller than the middle region 130B is capable of expanding to. This may be achieved, for example, by limiting the expansion size of the middle region 124C of the balloon 124 in FIGS. 8 and 9. If the physician determines that increasing the size of the middle region 130B of the scaffold 130 would be beneficial, the middle region 130B can be further expanded in diameter by either a different portion of the balloon (e.g., 124A or 124B) or by a second balloon catheter that inflates to a desired passage diameter.

Alternately, if the physician determines that the middle region 130B of the scaffold 130 was initially deployed with a diameter that is larger than desired, a second delivery catheter may be used to deliver a tubular spacer having a thickness that reduces the size of the passage through the middle region 130B. In one example, the tubular spacer may be a second scaffold 130, similar to the shunt initially deployed but deployed inside of the first shunt.

This ability to resize the scaffold 130 after delivery allows a physician to customize the amount of shunted fluid for each individual patient. It also allows the scaffold 130 to be modified at a later date if the patient's hemodynamic needs change.

In an alternate embodiment, the balloon catheter may include two or three separate, independently inflatable balloons that can be inflated to different sizes to achieve a similar hourglass shape. This may allow the physician to limit expansion of the middle of the scaffold 130 to a desired diameter while ensuring the ends of the scaffold 130 radially expand sufficiently to engage the surrounding tissue.

Another example shunt scaffold can be seen in FIG. 12 which illustrates a shunt scaffold 140 having a fenestrated body 142 defining a lumen 144 therethrough and anchoring features 146 and 148 on either side of the scaffold 140. Anchoring features 146 and 148 are embodied as a plurality of petals. The device 140 is shown with an optional cover 154 spanning between the various features of the scaffold 140. The cover 154 aids in anchoring the scaffold 140 and preventing leakage of fluids around the device. In one embodiment, the device is composed of a super elastic material such as Nitinol.

The scaffolds 130 or 140 (or those incorporated by reference) may further incorporate a flow control device that allows flow through the lumen in only one direction or allows flow through the lumen in only one direction and only if certain parameters are met. Alternatively, the device may further incorporate a flow control device that allows flow through the lumen in both directions, but only when certain parameters are met. The parameters that must be met in a first direction for fluid flow to be established may be the same or different than the parameters that must be met for fluid to flow in a second direction.

Adaptive shunt designs vary the flow profile based upon the pressure drop across the device. The principal of an adaptive shunt is such that the degree of shunting conferred by the device can be changed by intrinsic local conditions in response to a change in hemodynamic and/or anatomic parameters around which the device is placed. Such parameters may include, but are not limited to pressure, pressure gradient, absolute flow or flow gradients. The relationship between shunting and stimulus-response can be linear or nonlinear depending on the requirements of the individual situation. In addition to linearity/nonlinearity, thresholds can be built into such a shunt which function to begin or cease shunt at specific local conditions. These are ‘onset’ or ‘offset’ thresholds. In each case, for example, pressure or flow acts to change the effective shunt lumen size (open, close, other). The opening, if made highly nonlinear, can affect a ‘snap open’ or ‘snap closed’ result, effectively being a gating function of flow, pressure, or another regulated parameter.

The purpose of adaptive shunting is to protect organs or biologic tissues from pressure or flow damage. This protection may be conferred by limiting pressures at either the source or receiving end of the connection. For example, if the source of flow is the right atrium, this chamber cannot sustain prolonged elevated pressures and a “bleed off” shunt could be used to drop pressures which are approaching or exceeding a specified threshold value. Such a threshold value may be variable and inherently built into the device such that the pressure-flow relationship is linear, or nonlinear of any sort to accommodate physiologic benefit.

Adaptive shunts may thus be used as regulators for a pressure-flow relationship and would thus be made to function in an “autoregulatory mode”. This feature is useful to maintain healthy and safe pressures (for example) or other parameters by shunting flow (or other parameters) into lower resistance, or higher compliance chambers or channels.

In one example an adaptive shunt shunts more blood to the low-pressure chamber at higher pressures, feeding back on the source and lowering source pressure as it attempts to increase. Similarly, if pressure drops to lower levels the shunt will contract and shunt less blood from high-to-low pressure chamber, hence preventing the pressure to drop too low which would potentially dangerously reduce cardiac output.

Alternately, an adaptive shunt may reduce, limit, diffuse, or deflect blood flow to the low-pressure chamber at higher pressures. Creating a shunt from a high-pressure zone (e.g., in the aorta) to a low-pressure zone (e.g., in the pulmonary arter) may result in a high velocity jet of blood flow from one chamber to the other. If the high velocity jet hits the native vessel or chamber wall it will result in a high amount of shear stress on the tissue cells which may induce a biological response causing the formation of endothelial or scar tissue growth and ultimately vessel stenosis.

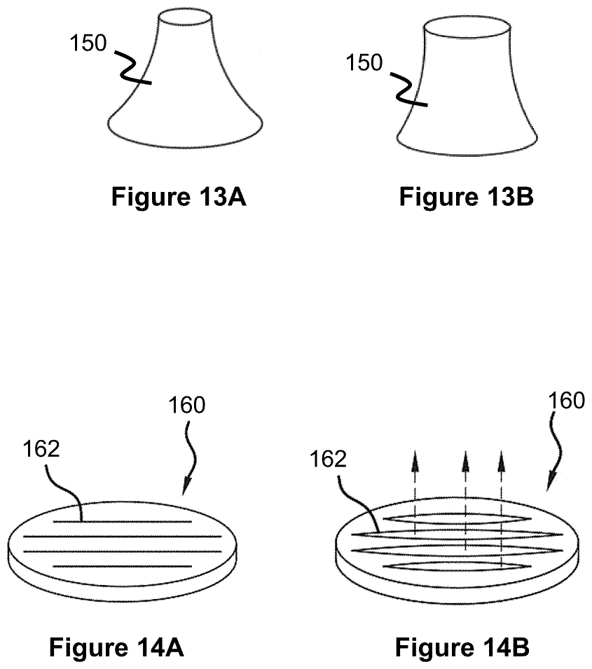

Stent-like devices, like the shunt scaffolds 130 or 140, can include valve mechanisms within their central passages to provide the previously discussed adaptive flow shunting. In one example, a conical member 150 composed of elastic material can be mounted within the central passage of a shunt device, moving from a relatively closed position (FIG. 13A) to a relatively open position (FIG. 13B). In another example, a relatively flat disc 160 can include a plurality of slits 162 that move from a closed position (FIG. 14A) to an open position (FIG. 14B). Other example mechanisms include spring mechanisms attached to a valve member, flaps, braided structures biased to a closed position that can be forced open, and other similar mechanisms.

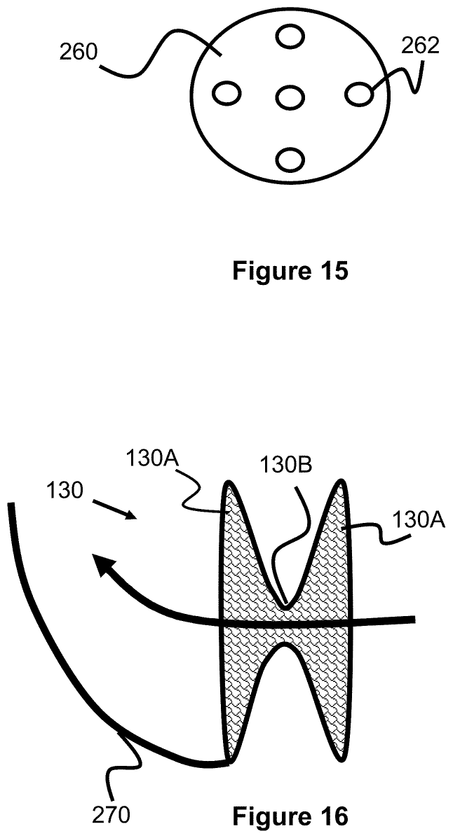

Stent-like devices, like the shunt devices 130 or 140, can also or alternately include a diffuser mechanism to prevent a high velocity jet to occur through the central passage of the shunt. For example, FIG. 15 illustrates a diffuser 260 which is composed of a flexible material that extends across the central passage of a shunt. The material includes a plurality of small apertures 262 that limit the amount of blood that can pass through and spread out the locations the blood passes through. In other words, the blood flow is diverted to several smaller locations that may decrease the shear stress on the tissue in the low-pressure chamber.

Stent-like devices, like the shunt devices 130 or 140, can also or alternately include a deflector that helps deflect the high velocity jet of blood in a direction in which it will not cause high shear stress on nearby tissue. For example, FIG. 16 illustrates a shunt 130 with a deflector 170 located on its end in the low-pressure chamber. The deflector 170 is preferably curved and therefore can be oriented in a desired off-axis direction relative to the shunt's internal passage.

In one embodiment, the deflector 170 can be a shape-memory loop of wire with a sheet of flexible material fixed across (e.g., a polymer sheet). The loop can be heat set to expand to the desired curved shape. In another example, the deflector 170 can be formed from a plurality of heat-set, shape memory wires woven into a mesh with an optional material sheet fixed over it. If the low-pressure chamber is a vessel, such as the pulmonary artery 20, the deflector 170 can be oriented so that the shunted blood is deflected in the same direction as natural blood flow (e.g., away from the pulmonary valve).

The deflector 170 can be located fully or partially within the passage of the shunt 130. In such a configuration, the deflector 170 may be a similar curved structure as discussed above or alternately a large rounded bump on only a portion of the inner circumference within the passage (i.e., the bump does not symmetrically extend entirely around the entire passage).

In another embodiment, some shunts of the present specification can be temporary and therefore slowly close over time. Example temporary shunts can be seen in PCT App. No. PCT/US2020/029013 filed Apr. 20, 2020 and entitled Temporary Shunt for Post-Operative Atrial Fibrillation, the contents of which are hereby incorporated by reference in its entirety.

For example, a piercing guidewire 106 can be used to make an initial puncture at a desired location and a balloon catheter can be positioned through the puncture and expanded. The resulting shunt passage can be allowed to heal naturally.

In another example of a temporary shunt, a plurality of smaller shunts can be created at a desired location. The smaller shunts may heal quicker than a single, larger shunt while providing a similar volume of blood to pass through relative to a larger shunt. In one example, the total area of all of the plurality of shunts is equal to a single shunt with an area of about 2-10 mm. In another example, 10-15 shunts can be created with a size of about 2 mm each. In another example, 5-10 shunts can be created with a size of about 3 mm each.

In another example of a temporary shunt, the previously described shunt support scaffolds can be composed of nondegradable materials or biodegradable materials that dissolve over a desired period of time. In one embodiment, the device is composed of a biodegradable material, such as a polymer of poly-L lactic acid (PLLA), tyrosine-derived polycarbonate, polylactic acid, or a bioabsorbable metal of magnesium or zinc. In this respect, the shunt scaffold will degrade after a period of time, allowing the shunt passage to heal. This may eliminate the need to block or cover the shunt in a later procedure, as discussed elsewhere in this specification.

To be clear, any of the shunts of the present specification can include any combination of valves, diffusers, and/or deflectors. For example, one shunt may have a valve and a deflector. Another shunt may have a diffuser and a deflector.

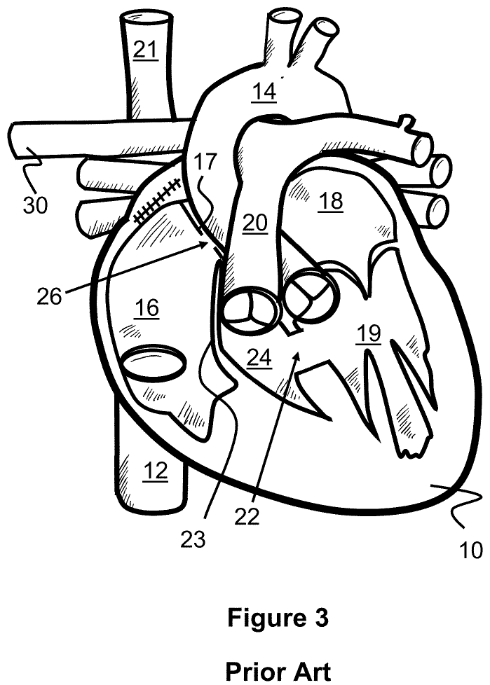

The original surgical Glenn procedure consists of disconnecting the superior vena cava 21 from the heart 10 and then connecting it to the right pulmonary artery 30, as seen in FIG. 3. The opening into the heart 10 of the superior vena cava 21 is then closed up. If the Blalock-Taussig shunt 29 is present from a prior Blalock-Taussig procedure, it is typically removed at this time. The result allows blood from the superior vena cava 21 to flow into the right pulmonary artery 30 so that it can be better oxygenated.

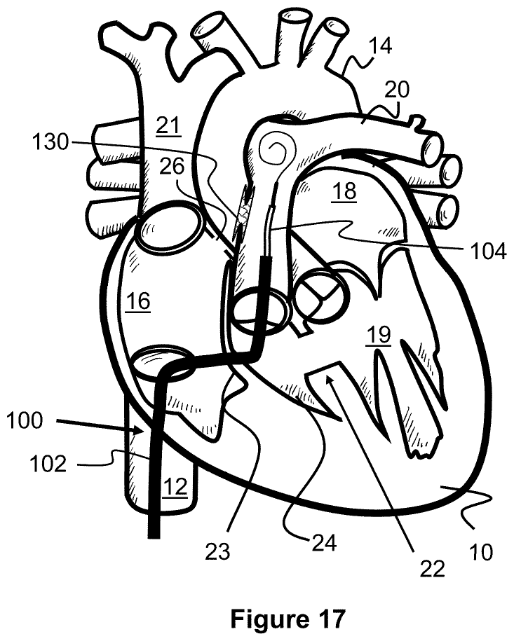

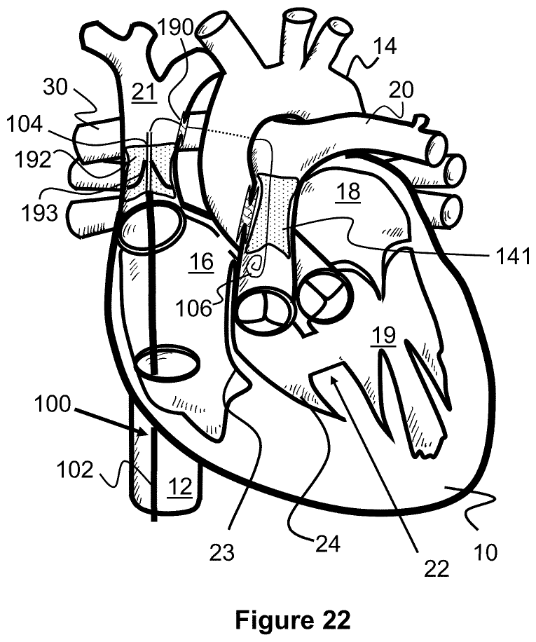

FIGS. 17-23 illustrate an alternate, transcatheter Glenn procedure in which the prior shunt scaffold 130 is closed, a new shunt passage is created with scaffold 190, and a one-way valve 192 is placed in the superior vena cava 21. Similar to the original Glenn procedure, this forces blood from an upper portion of the body back into the right pulmonary artery 30 so that it can bypass the heart and flow directly to the lungs.

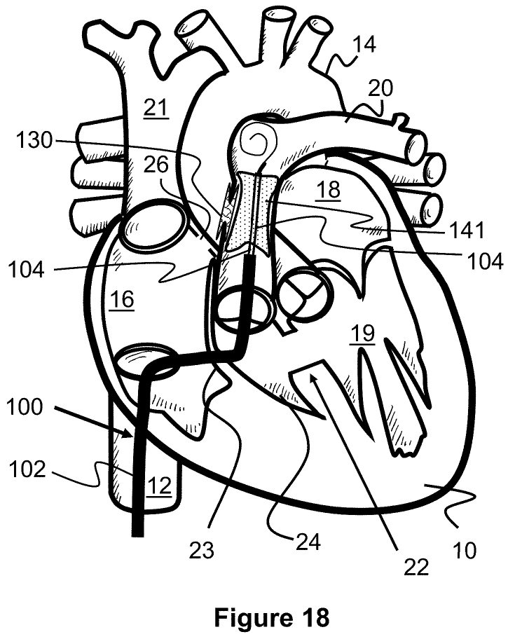

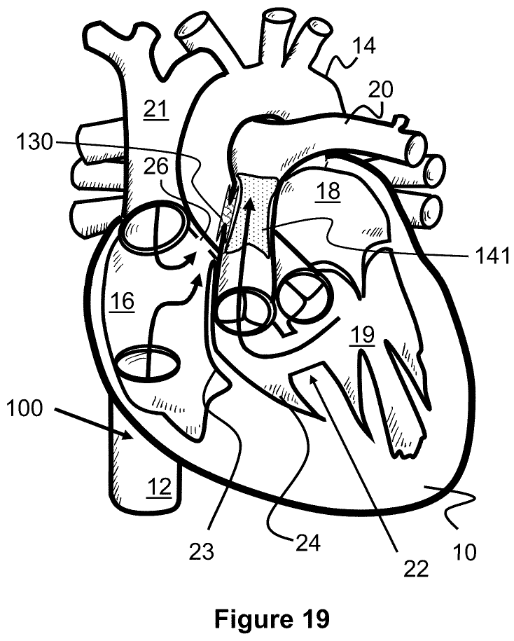

Turning to FIGS. 17 and 18, the shunt scaffold 130 is first closed if it is present in the patient. In one example, a delivery catheter 100 is advanced through the inferior vena cava 12, into the right atrium 16, through the tricuspid valve wall 23, through the pulmonary valve, and into the pulmonary artery 20. The outer catheter sheath 102 is withdrawn and a covered stent 141 is expanded from the inner catheter portion 106 over the shunt scaffold 130. The covered stent 141 may have a self-expanding or balloon expandable stent body (e.g., a laser cut metal tube or braided wire structure) and a polymer, fabric, or tissue covering that blocks the shunt passage through the shunt scaffold 130. The arrows of FIG. 19 illustrate the blood flow patterns once the shunt scaffold 130 has been closed.

Alternately, the delivery catheter 100 may instead deliver a self-expanding plug within the passage of the shunt scaffold 130. For example, this may include a smaller diameter shunt scaffold having a sheet or other material within its passage and configured to be deployed within the larger passage of the existing shunt scaffold (discussed in U.S. application Ser. No. 16/785,501 filed Feb. 7, 2020 and entitled Rivet Shunt and Method of Deployment, which was previously incorporated by reference).

In another alternate example, the shunt scaffold 130 may have a valve that initially remains open but can later be permanently closed by the delivery catheter 100. For example, the shunt scaffold 130 may include a heat sensitive adhesive that maintains the valve in an open position and can be melted by a heating element or similar heat generating component of the catheter 100 or separate radiofrequency device. Another example of a closure valve could be a system where the valve is help open by a physical lever. This physical lever could be actuated by an interventional catheter to close the valve. The lever could also be actuated via electrical contact and electrical actuation.

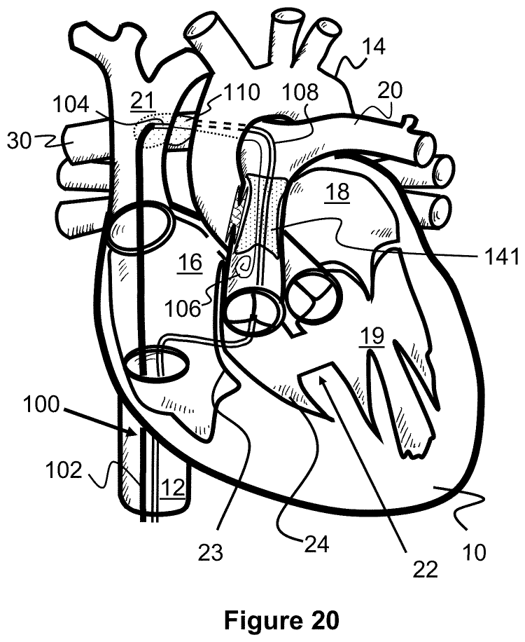

Next, a shunt is created between the superior vena cava 21 and the right pulmonary artery 30. In one example shown in FIG. 20, a target catheter 108 is first advanced up the inferior vena cave 12, into the right atrium 16, through the tricuspid valve wall 23, through the pulmonary valve, into the pulmonary artery 20, and into the right pulmonary artery 30. The previously described snare 110 is advanced out of the catheter 108 so that it is positioned near the overlap of the right pulmonary artery 30 and the superior vena cava 21. A tissue piercing guidewire 106 is also advanced through the inferior vena cava 12, into the right atrium 16, and into the superior vena cava 21. The guidewire 106 is angled towards and pierces through a wall of the superior vena cava 21 and the right pulmonary artery 30 (e.g., via RF ablation), and then can be advanced further into the pulmonary artery 20.

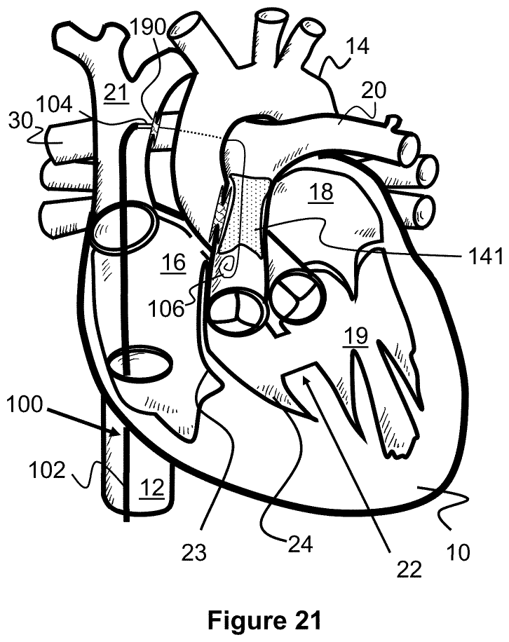

A delivery catheter 100 is then advanced over the guidewire 106 so that its distal tip passes into the right pulmonary artery 30. As seen in FIG. 21, a shunt scaffold 190 (similar to any of the previously described shunt scaffolds) is deployed, connecting the walls of the superior vena cava 21 and right pulmonary artery 30. For example, the outer catheter sheath 102 can be proximally withdrawn to allow the shunt scaffold 190 to self-expand from the inner catheter portion 104. Additionally, the target catheter 108 can be withdrawn.

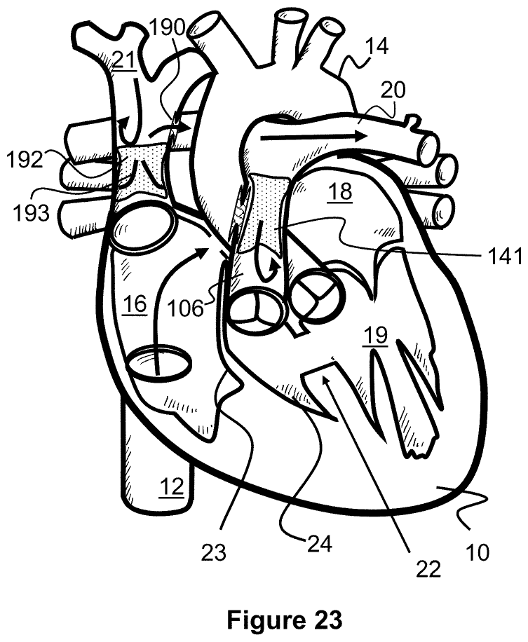

Next, a stent valve 192 is deployed within the superior vena cava 21, between the shunt scaffold 190 and the right atrium 16. The stent valve 192 includes a one-way valve mechanism 193 that allows blood to flow away from the right atrium 16, but not into the right atrium 16. In this respect, the stent valve 192 prevents blood from the upper portion of the body from returning to the heart 10 and instead redirects it to the right pulmonary artery 30 so that it can be oxygenated, as seen with the arrows of FIG. 23.

The stent valve 192 can be deployed from the same delivery catheter 100 that delivered shunt scaffold 190 (i.e., the catheter contains both devices) or a second delivery catheter 100 can be advanced over the guidewire 106 to deliver the stent valve 192. The one-way valve mechanism 193 can be any type of known valve mechanism, such as those described in FIGS. 13A, 13B, 14A, and 14B, artificial leaflets, or similar mechanisms that prevent blood flow in a first direction but allow blood flow in a second direction.

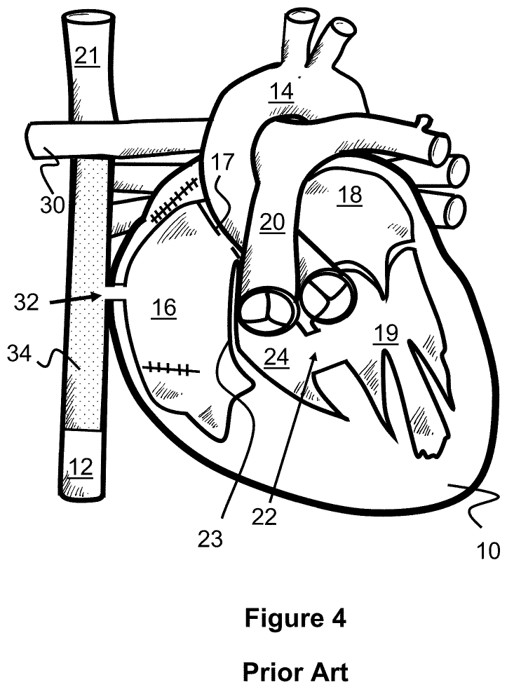

The original Fontan surgical procedure as shown in FIG. 4 consists of disconnecting the inferior vena cava 12 from the heart 10 and connecting it to the right pulmonary artery 30. Optionally, the shunt may include a passage or fenestration 32 into the right atrium 16 to allow some blood to flow directly back to the heart to help the lungs adjust to the extra blood flow. The original Fontan procedure is typically performed after the original Glenn procedure and therefore the superior vena cava 21 is typically already connected to the right pulmonary artery 30.

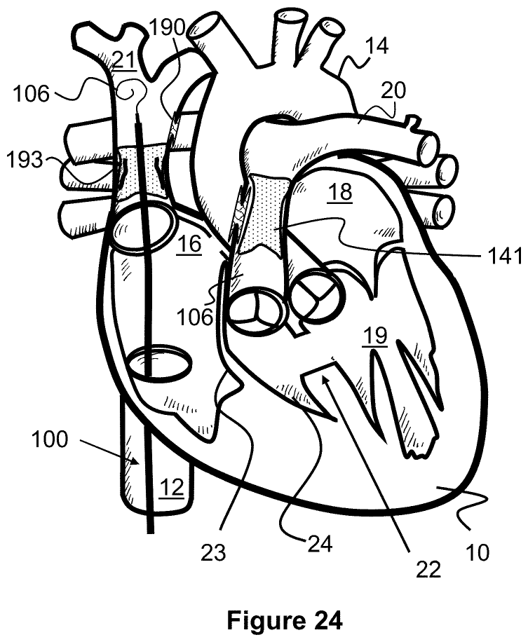

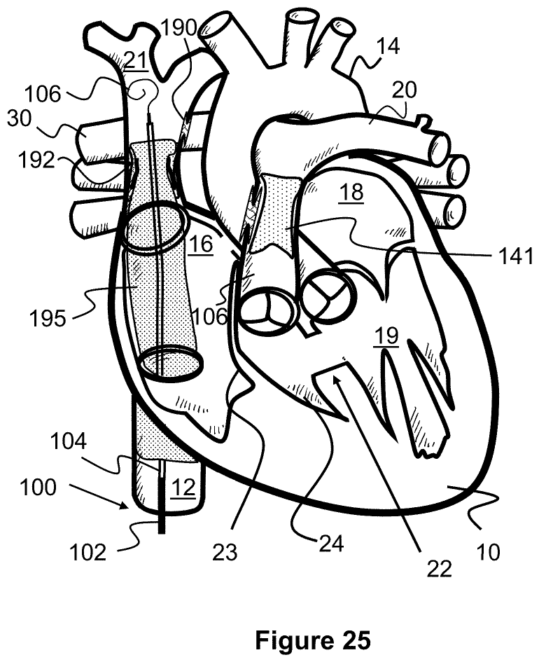

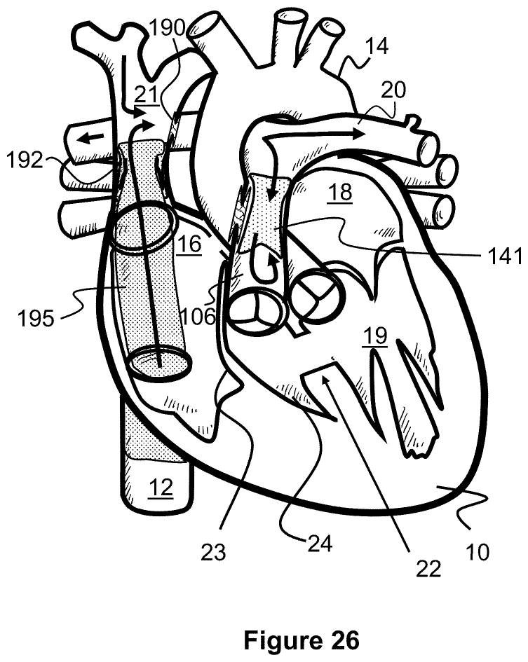

FIGS. 24-26 illustrate an alternate, transcatheter Fontan procedure that consists of deploying an elongated covered shunt or stent 195 between the inferior vena cava 12 and the superior vena cava 21. The top end of the covered stent 195 is preferably placed over the previously placed one-way stent valve 192, preventing it from closing, but below the stent scaffold 190 into the right pulmonary artery 30. The stent is designed to specifically anchor at the inferior vena cave and superior vena cava ostia. The SVC ostium is smaller, and the anchoring waist may have a diameter of 10-25 mm. The IVC ostium is larger, and the anchoring waist may have a diameter of 15-30 mm. The length of the covered synthetic graft may be 2-10 cm.

As seen in FIG. 24, a guidewire 106 and delivery catheter 100 are advanced through the inferior vena cava 12, through the right atrium 16, and into the superior vena cava 21. The outer catheter sheath 102 is proximally withdrawn to release the covered stent 196 so that it expands within the superior vena cava 21, right atrium 16, and inferior vena cava 12. Hence, a continuous passage is created between the inferior vena cava 12 and the superior vena cava 21, bypassing the right atrium 16 of the heart 10. This arrangement allows blood from both the upper and lower portions of the body to be directed through shunt scaffold 190, into the right pulmonary artery 30 so that it can be oxygenated.

The covered stent 196 can be one of a variety of known covered stents. For example, the covered stent 196 may include an underlying stent frame composed of a laser cut tube or braided metal wires, and may have a cover composed of a polymer (e.g., ePTFE) or tissue that blocks the flow of blood through the side of the stent framework. One example product (or a modified version thereof) that may be suitable for such a purpose is the AFX covered stent from Endologix that is otherwise used for treating abdominal aortic aneurysms.

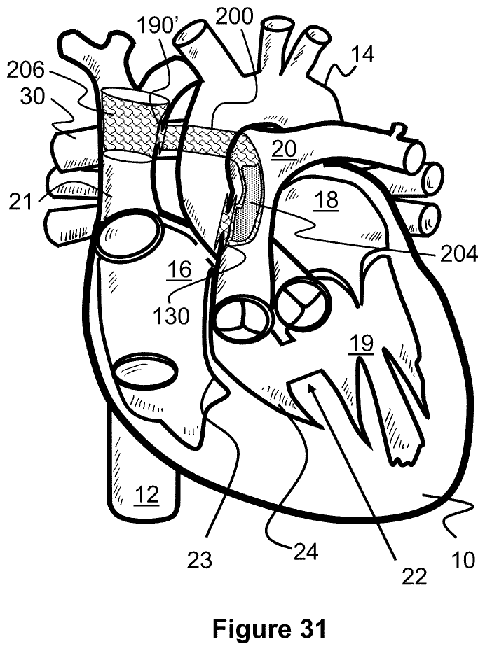

In another embodiment seen in FIG. 31, the covered stent 31 can be composed of an elongated tube 195A composed of material that prevents the flow of blood therethrough (e.g., textiles, polyurethane, PET, etc.). Optionally, the tube 195A may further include an internal support structure to help maintain its shape, such as one or more helical wires extending along its length. A top end of the tube 195A is connected to an expandable stent-like anchoring portion 195B that is configured to anchor in the superior vena cava 21. A bottom end of the tube 195A includes an expandable stent-like anchoring portion 195C that is configured to anchor in the inferior vena cava 12. In one example, the elongated covered shunt or stent 195 has a total length of about 10 cm, a diameter of the anchoring portion 195B of about 2 cm, and a diameter of the anchoring portion 195C of about 2 cm.

In some embodiments, the previously described methods can be modified to prepare for later procedures. For example, the transcatheter Blalock-Taussig procedure can be modified to allow the transcatheter Glenn procedure to be more easily performed.

In a specific example shown in FIG. 27, shunt support scaffolds 130 and 190′ can be placed during an initial transcatheter Blalock-Taussig procedure. However, the shunt 190′ includes a sheet 199 (e.g., textile, polyurethane, silicone, PET, etc.) covering its passage and therefore leaving it initially closed (see FIG. 28). In a later procedure, a balloon catheter can be inserted through the sheet 199 (either through an existing aperture in the sheet 199 or via a guidewire 106 that has pierced the sheet) and expanded to open up the shunt scaffold 190′.

In this respect, placement of both shunt scaffolds 130 and 190′ can occur during one initial procedure by advancing a guidewire 106 into the superior vena cava 21, into the right pulmonary artery 20, into the trunk of the pulmonary artery 20, and finally into the aorta 14. A single delivery catheter 100 or two separate delivery catheters can be advanced over the guidewire 106 to deliver the shunt scaffolds 130 and 190′ in their respective locations. Hence, a simple balloon catheter procedure can be used to open shunt scaffold 190′ at the time of the transcatheter Glenn procedure.

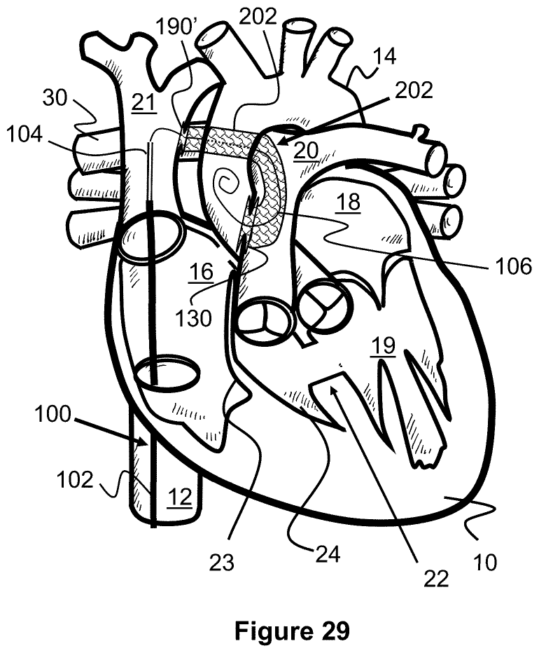

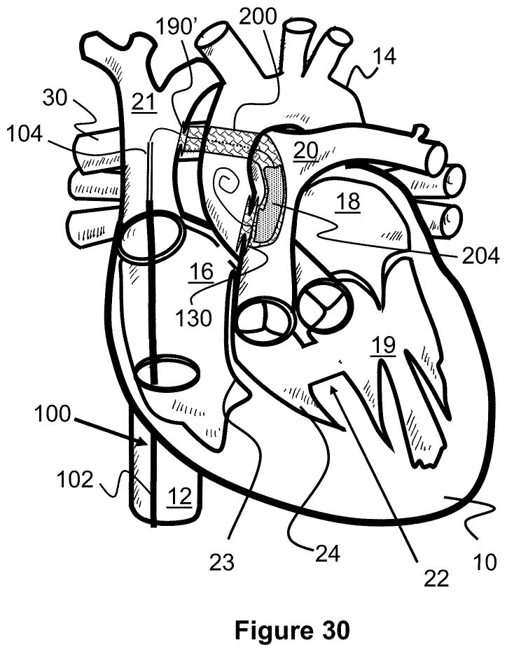

FIGS. 29 and 30 illustrate a similar Blalock-Taussig procedure that facilitates a later Glenn procedure by using an elongated shunt support structure 200. The elongated shunt support structure 200 preferably is comprised of a first shunt support scaffold 130, a second shunt support scaffold 190′, and a blood-permeable tubular portion 202 through which blood can pass. The shunt structures 130 and 190′ are similar to those previously described in this specification (including a sheet 199 in structure 190′). The tubular portion 202 is connected to both scaffolds 130 and 190, and can be composed of a permeable structure, such as a laser-cut tubular structure with a plurality of openings or a braided wire tubular structure with a plurality of openings. The support structure 200 can be deployed in a manner similar to that described for the scaffolds 130, 190′ of FIG. 27, except as one continuous device. In one embodiment, the elongated support structure 200 is about 3-1 cm in length.

When initially deployed (FIG. 29), the support structure 200 allows blood to pass through the side walls of the tubular structure 202 and then through the opening of the support scaffold 130, while the sheet 199 in the shunt scaffold 190′ blocks blood flow between the inferior vena cava 21 and the right pulmonary artery 30.

During the later Glenn procedure, a blocking device 204 is delivered within the support structure 200, near the scaffold 130 to block blood flow from the pulmonary artery 20 into the aorta 20, as seen in FIG. 30. The sheet 199 in scaffold 190′ can then be opened via balloon catheter to allow the blood flow from the superior vena cava 21 to the aorta 20. The blocking device 204 can be a stent-like device having an expandable, rigid structure (e.g., laser-cut tube or braided wire tube) with an outer covering on its sides and at least one end that blocks the flow of blood (e.g., textile, polyurethane, silicone, PET, etc.). Alternately, the blocking device 204 can be an expandable foam structure. The support structure 200 and/or the blocking device 204 can include anchoring elements that help maintain the placement of the blocking device 204, such as hooks or barbs.

FIG. 31 illustrates a Blalock-Taussig procedure and later Glenn procedure, as described for FIGS. 29 and 30, but further includes a landing zone portion 206 that is connected to or integral with the support structure 200. The landing zone portion 206 provides an area that the previously described covered stent 195 can attach to during a transcatheter Fontan procedure. The landing zone portion 206 can have a stent-like structure (e.g., laser-cut tube or braided tubular structure) configured to anchor within the superior vena cava 21 with a side opening into the passage of scaffold 190′, a top opening, and a bottom opening. The bottom opening is preferably positioned below the scaffold 190′ and the lower portion around the bottom opening may include anchoring elements (e.g., hoops or barbs) that facilitate anchoring of the covered stent 195 during the transcatheter Fontan procedure.

In any of the procedures described in this specification that use both a target catheter and a delivery catheter, it should be understood that their respective approaches and positions can be reversed. In other words, while the target catheter may be described as being positioned in a first location and the target catheter positioned in a second location, these locations can be reversed, allowing for alternate approaches and delivery methods.

The present invention also contemplates kits containing at least some of the devices necessary to perform the procedures described in this specification. For example, the transcatheter Blalock-Taussig kit may include 1) a target catheter 108 configured to extend into the aorta 14 or aortic arch (e.g., >85 cm in length and <6Fr French), 2) a tissue piercing guidewire 106 (e.g., RF guidewire) configured to extend into the pulmonary artery 20 and the aorta 14 (e.g., 200-350 cm in length and 0.014″−0.035″OD), 3) a delivery catheter 100 configured to extend into the pulmonary artery 20 and the aorta 14 (e.g., >85 cm in length and <6 French), and 4) a shunt scaffold 130 configured to create a shunt between the pulmonary artery 20 and the aorta 14 (e.g., a device diameter of about 2-10 mm, and a shunt passage of about 2-10 mm in diameter and 5-10 mm in length).

In another example, the transcatheter Glenn procedure kit may optionally include 1) a first delivery catheter 100 configured to extend into the pulmonary artery 20 (e.g., >85 cm length, <6 French) and 2) a covered stent sized to anchor within the pulmonary artery 20 and cover a previously deployed shunt scaffold 130 from the previously described transcatheter Blalock-Taussig procedure. For the second portion of the Glenn procedure, the kit may also include 3) a target catheter 108 configured to extend into the right pulmonary artery 30 (e.g., >85 cm in length and <6 French), 4) a tissue piercing guidewire 106 (e.g., RF guidewire) configured to extend into the right pulmonary artery 30 from the superior vena cava 21 (e.g., 200-350 cm in length and 0.014″-0.035″OD), 5) a delivery catheter 100 configured to extend into the superior vena cava 21 and the right pulmonary artery 30 (e.g., >85 cm in length and <6 French), 6) a shunt scaffold 130 configured to create a shunt between the superior vena cava 21 and the right pulmonary artery 30 (e.g., a device diameter of about 2-12 mm and a shunt passage of about 2-12 mm in diameter and 3-10 mm in length), 7) a second delivery catheter 100 that is configured to extend into the superior vena cava 21 (e.g., >80 cm length, <6 French), and 8) a one-way stent valve 192 that is configured to anchor in a superior vena cava 21 (e.g., 5-25 mm in diameter).

In another example, the transcatheter Fontan kit may include 1) a delivery catheter 100 configured to extend into the superior vena cava 21, and 2) a covered stent 195 that is configured to be anchored in the superior vena cava 21, extend through the right atrium 16, and further be anchored in the inferior vena cava 12 (e.g., 60-100 mm in length, and 5-20 mm in diameter).

In another example, the previously described transcatheter Blalock-Taussig kit, Glenn kit, and Fontan kit may all be included in a single kit for performing all procedures.

While the figures and the specification describe the present invention in the context of a heart with tricuspid atresia, it should be understood that hearts and circulatory systems with other defects, such as pulmonary atresia, pulmonary stenosis, Tetralogy of Fallot, and hypoplastic left heart syndrome can also be used with one or more of the methods of the present invention. In other words, it is not necessarily intended to limit treatment with the present invention to only tricuspid atresia.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A method for treating a heart defect, comprising:
advancing a delivery catheter through a wall of a pulmonary artery and a wall of an aorta; and,
delivering a shunt scaffold within the wall of the pulmonary artery and the wall of the aorta to create a shunt between the pulmonary artery and the aorta.

2. The method of claim 1, wherein advancing a delivery catheter is preceded by advancing a guidewire from the pulmonary artery into the aorta.

3. The method of claim 2, wherein advancing the guidewire from the pulmonary artery into the aorta is preceded by advancing a target catheter into the aorta and deploying a snare at a location adjacent to the pulmonary artery; wherein the snare is configured to resist puncture by the guidewire.

4. The method of claim 3, wherein the delivery catheter is advanced over the guidewire, into the pulmonary artery, and into the aorta.

5. The method of claim 4, wherein the guidewire is positioned through a heart wall formed from an undeveloped tricuspid valve.

6. A method for treating a heart defect, comprising:
advancing a delivery catheter through a wall of a superior vena cava and a wall of a right pulmonary artery; and,
delivering a shunt scaffold within the wall of the superior vena cava and the wall of the right pulmonary artery.

7. The method of claim 6, wherein advancing the delivery catheter through the wall of the superior vena cava and the wall of the right pulmonary artery is proceeded by advancing a target catheter into the right pulmonary artery and deploying a snare from a distal end of the target catheter, adjacent to the superior vena cava.

8. The method of claim 7, wherein advancing the target catheter into the right pulmonary artery is preceded by advancing a guidewire into the superior vena cava and into the right pulmonary artery; and advancing the delivery catheter over the guidewire and partially into the right pulmonary artery.

9. The method of claim 8, further comprising delivering a stent containing a one-way valve in the superior vena cava between the shunt scaffold and a right atrium of a heart, wherein the one-way valve is configured to allow blood to only flow in a direction away from the right atrium.

10. The method of claim 8, wherein advancing the guidewire into the superior vena cava is preceded by delivering a covered stent within an aorta to cover and close a shunt between the aorta and a pulmonary artery trunk.

11. A method for treating a heart defect, comprising:
advancing a distal end of a delivery catheter into a superior vena cava; and,
delivering a covered stent between the superior vena cava, through the right atrium, and into the inferior vena cava.

12. The method of claim 11, wherein the covered stent is deployed over a one-way stent valve located in the superior vena cava.

13. The method of claim 11, wherein the covered stent is deployed prior to a shunt connecting the superior vena cava and a right pulmonary artery; wherein the shunt remains substantially uncovered by the covered stent.

14. A method for treating a heart defect, comprising:
advancing a first delivery catheter through a wall of a pulmonary artery trunk and a wall of an aorta;
delivering a first shunt scaffold within the wall of the pulmonary artery trunk and the wall of the aorta to create a shunt between the pulmonary artery trunk and the aorta;
advancing a second delivery catheter into the pulmonary artery trunk and closing a passage through the first shunt scaffold;
advancing a third delivery catheter through a wall of a superior vena cava and a wall of a right pulmonary artery;
delivering a second shunt scaffold within the wall of the superior vena cava and the wall of the right pulmonary artery;
delivering a stent valve containing a one-way valve mechanism into the superior vena cava between the second shunt scaffold and a right atrium;
advancing a fourth delivery catheter into the superior vena cava;
delivering a covered stent within the superior vena cava, the right atrium, and the inferior vena cava, such that the second stent scaffold remains substantially uncovered and the stent valve remains substantially covered.

15-22. (canceled)
